(19) United States
(12) Patent Application Publication
(10) Pub. No.: US 2023/0175015 A1
(43) Pub. Date: Jun. 8, 2023

(54) IMMUNOSUPPRESSIVE AGENTS AND VIRAL DELIVERY RE-DOSING METHODS FOR GENE THERAPY

(71) Applicant: MYOGENE BIO LLC, San Diego, CA (US)

(72) Inventors: Courtney YOUNG, San Diego, CA (US); Melissa SPENCER, San Diego, CA (US); April PYLE, San Diego, CA (US)

(21) Appl. No.: 17/998,454

(22) PCT Filed: May 12, 2021
(86) PCT No.: PCT/US2021/031994

§ 371 Date: Nov. 10, 2022

Related U.S. Application Data
(60) Provisional application No. 63/023,767, filed on May 12, 2020.

(51) Int. Cl. C12N 15/86; C12N 9/22; C12N 15/11; A61K 31/573; A61K 31/436; A61K 39/395; A61K 38/20; A61K 31/519; A61K 31/5377; A61K 31/225; A61K 38/17

(52) U.S. Cl. CPC C12N 15/86; C12N 9/22; C12N 15/11; A61K 31/573; A61K 31/436; A61K 39/3955; A61K 38/2013; A61K 31/519; A61K 31/5377; A61K 31/225; A61K 38/1774; C12N 2750/14143; C12N 2750/14171; C12N 2310/20; C12N 2800/80

(57) ABSTRACT

Provided are methods related generally to the fields of molecular biology and virology, in particular the use of immunosuppressive agents and methods for treating using gene therapy, notably Duchenne muscular dystrophy.

### CROSS-REFERENCE

This application claims the benefit of priority of U.S. Provisional Patent Application Ser. No. 63/023,767 filed May 12, 2020, the disclosure of which is incorporated by reference in its entirety for all purposes.

### STATEMENT OF GOVERNMENT SUPPORT

This invention was made with government support under 1R44AR075469-01A1, awarded by the National Institutes of Health. The government has certain rights in the invention.

### FIELD

The present disclosure generally relates to the fields of molecular biology and virology and, in particular, the use of immunosuppressive agents and viral re-dosing methods for gene therapy, notably Duchenne muscular dystrophy.

### BACKGROUND

Major advances in gene therapy have been achieved by using viruses to deliver therapeutic genetic material. The adeno-associated virus (AAV) has attracted considerable attention as a viral vector for gene therapy due to its low immunogenicity and ability to effectively transduce non-dividing cells. AAV has been shown to infect various cell and tissue types. Significant progress has been made over the last decade to adapt this viral system for human gene therapy. The first FDA-approved AAV gene therapy drug, Glybera, was approved in 2012. Certain serotypes of AAV have tropism for skeletal muscle and heart tissue. These are currently being used in human clinical trials.

In its normal “wild type” form, AAV DNA is packaged into the viral capsid as a single-stranded molecule about 4600 nucleotides (nt) in length. Following viral infection, the molecular machinery of the cell converts the single-stranded DNA into a double-stranded form. Cellular enzymes can transcribe only this double-stranded DNA form into RNA, then translated it into polypeptides by additional cellular pathways.

Recombinant adeno-associated virus (rAAV) vectors have been used successfully for in vivo gene transfer in numerous pre-clinical animal models of human disease, including restoration of vision in patients with Leber's congenital amaurosis by retinal gene transfer and hemophilia B by hepatic gene therapy. This vector has a comparatively low immune profile, eliciting only limited inflammatory responses and, in some cases, even directing immune tolerance to transgene products.

Muscular wasting diseases, such as muscular dystrophies, are a group of degenerative diseases that culminate in progressive skeletal muscle wasting leading to muscle weakness, a high incidence of bone fracture, wheelchair dependence, and, in some cases, death. Of the muscular dystrophies, Duchenne muscular dystrophy is the most severe and most widely recognized. Another muscular wasting disease that shows similar symptoms, although less severe than Duchenne muscular dystrophy, is Becker muscular dystrophy. Even though the defective dystrophin gene causing both Duchenne muscular dystrophy and Becker muscular dystrophy has been known for over 20 years, a cure is still lacking.

AAV gene therapy can be used to carry a CRISPR/Cas9 system for Duchenne muscular dystrophy (WO Publication 2017139505). An early clinical trial in Duchenne muscular dystrophy used an AAV-mini-dystrophin transgene. No dystrophin production was observed, likely due to rejection by dystrophin reactive T cells. This study used a CMV promoter to allow the mini-dystrophin to be expressed in antigen-presenting cells. Current clinical trials are using muscle-specific promoters.

Therapeutic efficacy loss has occurred due to immune responses to the virus or transgene, which can be mitigated by immunosuppression with prednisolone. While this intervention mitigated rejection of the virus and the transgene, it is likely not sufficient to allow subsequent re-dose of the vector. What is absent from the prior art is an immunosuppression protocol that would repress the immune system and allow for AAV-CRISPR re-dosing. Therefore, developing an effective immunosuppressive protocol would increase the efficacy and safety of AAV gene therapies for muscle tissue and provide a method for treating Duchenne muscular dystrophy.

### SUMMARY

The present disclosure provides a method for viral delivery of a nucleic acid in a patient in need thereof, comprising administering to the patient one or more doses of a particle comprising a recombinant adeno-associated viral (rAAV) nucleic acid vector, the vector comprising a polynucleotide that comprises a nucleic acid segment, wherein administering the particle to the patient results in deletion of a segment of a mutant gene in the patient.

The present disclosure also provides a method for treating muscular dystrophy in a patient in need thereof, comprising administering to the patient one or more doses of a particle comprising a recombinant adeno-associated viral (rAAV) nucleic acid vector. The vector comprises a polynucleotide that comprises a first nucleotide sequence that encodes a class 2 CRISPR/Cas endonuclease and one or more second nucleotide sequences from which are transcribed a first and a second CRISPR/Cas9 guide RNA. The first CRISPR/Cas9 guide RNA comprises a first guide sequence that hybridizes to a first target sequence within intron 44 of the mutant dystrophin gene in the patient. The second CRISPR/Cas9 guide RNA comprises a second guide sequence that hybridizes to a second target sequence within intron 55 of the mutant dystrophin gene in the patient. Administering the particle results in a deletion of a greater than 330 kb region of the mutant dystrophin gene comprising exons 45-55 in the patient.

The present disclosure further provides a recombinant adeno-associated viral (rAAV) particle comprising an rAAV nucleic acid vector, the vector comprising a polynucleotide that comprises a first nucleotide sequence that encodes a class 2 CRISPR/Cas endonuclease and one or more second nucleotide sequences, from which are transcribed a first and a second CRISPR/Cas9 guide RNA, wherein the first CRISPR/Cas9 guide RNA comprises a first guide sequence that hybridizes to a first target sequence. The second CRISPR/Cas9 guide RNA comprises a second guide sequence that hybridizes to a second target sequence within intron 55 of the mutant dystrophin gene.

For promoting an understanding of the principles of the disclosure, reference will now be made to the embodiments or examples illustrated in the drawings, and specific language will be used to describe the same. It will be understood that no limitation of the scope of the disclosure is thereby intended. Any alterations and further modifications in the described embodiments and any further applications of the principles of the disclosure as described herein are contemplated as would normally occur to one of ordinary skill in the art to which the disclosure relates.

### DESCRIPTION OF THE DRAWINGS

The following drawings form part of the present specification and are included to demonstrate certain aspects of the disclosure. The disclosure may be better understood by reference to the following description taken in conjunction with the accompanying drawings, in which like reference numerals identify like elements.

FIG. 1 shows a schematic depicting the timeline (in days). The highlighted range with “IS” representing when the immunosuppressive (IS) protocols were administered. Each dose of AAV has a different cargo, so the effectiveness of each dose can be assessed.

FIG. 2A shows the first dose expression cassette containing Green Fluorescent Protein (GFP) driven by a cytomegalovirus (CMV) promoter. The second dose expression cassette contains gRNA sequences driven by a human RNA polymerase III promoter, U6, and mCherry driven by a cytomegalovirus (CMV) promoter.

FIG. 2B shows one expression cassette for CRISPR for DMD. SpCas9 was driven by a cytomegalovirus (CMV) promoter or a Ck8 promoter. The second expression cassette contains gRNA sequences (SEQ ID NO:1 and SEQ ID NO:2) driven by a human RNA polymerase III promoter, U6, and mCherry driven by a cytomegalovirus (CMV) promoter. Expression cassettes are injected together.

FIGS. 3A-C show the assessment of AAV9 redosing in hDMD de145 mdx mice. Representative mosaic images of the heart (FIG. 3A), triceps (FIG. 3B), and tibialis anterior (FIG. 3C) sections stained with laminin (grey) to mark muscle cells and imaged for GFP as a readout of the first AAV injection and mCherry as a readout of the second injection (redosing). Images for mice treated with all four drugs, without CTLA4Ig (−), anti-CD20 (−), and sirolimus (−) are shown. With all drugs or without CTLA4Ig, mCherry expression is observed, demonstrating this regimen allowed AAV redosing. Without an anti-CD20 antibody or sirolimus, no mCherry is seen.

FIGS. 4A-C show the assessment of AAV9 redosing as measured by Western blotting in hDMD de145 mdx mice. Western blotting for GFP and mCherry in lysates from hDMD de145 mdx mice from isolated hearts (FIG. 4A), triceps (FIG. 4B), and liver (FIG. 4C). Positive (pos) and negative (neg) controls are shown on the heart and liver blots. α-Actinin is shown below each muscle blot for loading. No mCherry was observed when the CD20 antibody and sirolimus were removed. Redosing of mCherry expression was shown with all drugs or without CTLA4Ig.

FIGS. 5A-B show the assessment of AAV redosing in mice. Representative mosaic images of the heart (A) and triceps (B) sections stained with laminin to mark muscle cells and imaged for GFP as a readout of the first AAV injection and mCherry as a readout of the second injection (redosing). Images for mice treated with different combinations of anti-CD20 antibody (CD20), sirolimus (siro), prednisone (pred), and IL2 complex (IL2C) are shown. Control groups of prednisone only, no immune suppression and untreated are shown in the heart staining.

FIGS. 6A-B show the assessment of AAV redosing as measured by Western blotting in mdx mice. Western blotting for GFP and mCherry in lysates from mdx mice from isolated hearts (FIG. 6A) and triceps (FIG. 6B) muscles from FIGS. 5A and B, respectively. Single-injection GFP/mCherry only controls are shown on the triceps blot.

FIGS. 7A-C show the assessment of AAV redosing in mdx mice. Representative mosaic images of the heart (FIG. 7A) and triceps (FIG. 7B) sections stained with laminin to mark muscle cells and imaged for GFP as a readout of the first AAV injection and mCherry as a readout of the second injection (redosing). Images for mice treated with different combinations of anti-CD20 antibody (CD20), sirolimus (siro), prednisone (pred), anti-CS antibody (C5), anti-CD79b antibody (CD79b), anti-CD19 antibody (CD19), ruxolitinib (ruxo), and ibrutinib (ibrut) are shown. Western blotting in selected heart samples (FIG. 7C) for GFP and mCherry. GAPDH or Ponceau is shown for loading. Single-injection GFP/mCherry only controls are shown. Mouse #3 from each group was kept for an additional month without immune suppression.

FIGS. 8A-B. show the assessment of dystrophin after redosing AAV-CRISPR. Representative dystrophin immunostaining (FIG. 8A) and Western blotting (FIG. 8B) in the heart after a single or double injection of dual vector AAV-CRISPR (containing Ck8-Cas9 and gRNAs to delete exons 45-55 and restore dystrophin) in hDMD de145 mdx pups also given anti-CD20 antibody (CD20), sirolimus (siro) and prednisone (pred) immune suppression. An untreated control heart is shown for immunostaining. The percent of wild-type dystrophin by Western blot was quantified using an hDMD wild-type (wt) standard curve shown in parentheses. The average dystrophin level was 60% after a single injection (n=3) and 45% after double injections (n=2). Dystrophin lacking exons 45-55 is shifted compared to wild-type size on the blot. Alpha-actinin is shown for loading.

### DETAILED DESCRIPTION

The present disclosure provides a method for treating muscular dystrophy in a patient in need thereof, comprising administering to the patient one or more doses of a particle comprising a recombinant adeno-associated viral (rAAV) nucleic acid vector. In certain embodiments, the vector comprises a polynucleotide that comprises a first nucleotide sequence that encodes a class 2 CRISPR/Cas endonuclease and one or more second nucleotide sequences from which are transcribed a first and a second CRISPR/Cas9 guide RNA. In certain embodiments, the first CRISPR/Cas9 guide RNA comprises a first guide sequence that hybridizes to a first target sequence, for example, within intron 44 of the mutant dystrophin gene in the patient. In certain embodiments, the second CRISPR/Cas9 guide RNA comprises a second guide sequence that hybridizes to a second target sequence, for example, within intron 55 of the mutant dystrophin gene in the patient. In certain embodiments, administering the particle results in a deletion of a greater than 330 kb region of the mutant dystrophin gene comprising exons 45-55 in the patient.

In further embodiments, the muscular dystrophy is chosen from Duchenne muscular dystrophy, Becker muscular dystrophy, limb girdle muscular dystrophy, congenital muscular dystrophy, facioscapulohumeral muscular dystrophy, myotonic muscular dystrophy, oculopharyngeal muscular dystrophy, distal muscular dystrophy, and Emery-Dreifuss muscular dystrophy.

The symptoms of Duchenne muscular dystrophy include muscle weakness which usually begins around the age of four in boys and worsens quickly. Typically, muscle loss occurs first in the thighs and pelvis, followed by those of the arms. This muscle loss can result in trouble standing up. Most are unable to walk by the age of 12. Affected muscles may look larger due to increased fat content. Scoliosis is also common. Some may have an intellectual disability. Females with a single copy of the defective gene may show mild symptoms.

The disorder is X-linked recessive. About two-thirds of cases are inherited from a person's mother, while one-third of cases are due to a new mutation. It is caused by a mutation, typically out-of-frame, in the DMD gene encoding the protein dystrophin. Dystrophin maintains the muscle fiber's cell membrane. Genetic testing can often diagnose at birth. Those affected also have a high level of creatine kinase in their blood.

Although there is no known cure, physical therapy, braces, and corrective surgery may help with some symptoms. Assisted ventilation may assist those with weakness of breathing muscles. Medications used include corticosteroids to slow muscle degeneration, reduce inflammation, and cardiac drugs to help with the heart effect.

DMD affects about one in 5,000 males at birth. It is the most common type of muscular dystrophy. The average life expectancy is 26; however, with excellent care, some may live into their 30s or 40s.

Previous studies have tested several immunosuppressive agents in animal models to allow for AAV re-dosing, such as CD4 antibody, CTLA4-Ig/CD40, a rituximab and sirolimus combination, and a rituximab and cyclosporin combination. Other strategies to induce tolerance through T regulatory cells have shown improved efficacy and dampened immune responses. Currently, a human clinical trial is ongoing involving the redosing of intramuscular AAV using rituximab and sirolimus. However, no studies have investigated re-dosing when CRISPR/Cas9 is the AAV cargo. AAV mediated delivery of Cas9 will be evaluated since the Cas9 protein will continue to be expressed in post-mitotic muscle, potentially, for years, presenting an increased potential for an immune response. Since pre-existing anti-Cas9 antibodies have been observed in healthy adults, redosing will be investigated.

In some embodiments, the novel rAAV nucleic acid vectors express constructs and infectious virions and viral particles comprising them, as disclosed herein.

In some embodiments, the disclosure provides rAAV particles, including those derived from one or more serotypes as known in the art (including, for example, those chosen from AAV1, AAV2, AAV3, AAV4, AAV5, AAV6, AAV7, AAVS, AAV9, AAV10, AAV11, and AAV12).

The present disclosure also concerns rAAV nucleic acid vectors. The nucleic acid segment further comprises a promoter, an enhancer, a post-transcriptional regulatory sequence, a polyadenylation signal, or any combination thereof, which is operably linked to the nucleic acid segment that encodes the selected polynucleotide of interest.

In certain embodiments, the nucleic acid segments cloned into the novel rAAV expression vectors described herein will express or encode one or more polypeptides, peptides, ribozymes, peptide nucleic acids, siRNAs, RNAi, guide RNAs (gRNAs), antisense oligonucleotides, antisense polynucleotides, antibodies, antigen-binding fragments, or any combination thereof.

Examples of suitable therapeutic agents include, but are not limited to, one or more agonists, antagonists, anti-apoptosis factors, inhibitors, receptors, cytokines, cytotoxins, erythropoietic agents, glycoproteins, growth factors, growth factor receptors, hormones, hormone receptors, interferons, interleukins, interleukin receptors, nerve growth factors, neuroactive peptides, neuroactive peptide receptors, proteases, protease inhibitors, protein decarboxylases, protein kinases, protein kinase inhibitors, enzymes, receptor binding proteins, transport proteins or one or more inhibitors thereof, serotonin receptors, or one or more uptake inhibitors thereof, serpins, serpin receptors, tumor suppressors, activators, antibodies and fragments thereof, diagnostic molecules, chemotherapeutic agents, cytotoxins, or any combination thereof.

The rAAV nucleic acid vectors disclosure may be contained within a virion or viral particle having a serotype that is chosen from AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12), or any other serotype as known to one of ordinary skill in the viral arts. “Identical serotype,” as used herein, refers to the AAV having the same serotype number.

In related embodiments, the disclosure further provides populations and pluralities of rAAV nucleic acid vectors, virions, infectious viral particles, or host cells that comprise one or more nucleic acid segments that, for example, encode an autoimmune disease therapeutic agent.

The disclosure further provides compositions and formulations that comprise one or more of the proteins, nucleic acid segments, viral vectors, host cells, or viral particles disclosed herein, together with one or more pharmaceutically acceptable buffers, diluents, or excipients. Such compositions may be included in one or more diagnostic or therapeutic kits for diagnosing, preventing, treating, or ameliorating one or more symptoms of a mammalian disease, particularly for delivering a therapeutic agent for treating Duchenne muscular dystrophy in a patient.

The disclosure also provides a method of transducing a population of mammalian cells. Generally, the method comprises introducing into one or more cells of the population a composition comprising an effective amount of one or more of the rAAV nucleic acid vectors, wherein the one or more rAAV vector-based gene therapy constructs is administered once or twice or multiple times during a treatment protocol for a gene therapy treatable disorder. In some embodiments, one or more rAAV vector-based gene therapy constructs are formulated with one or more additional immunosuppressive agents.

In some embodiments, isolated nucleic acid segments encode one or more of the rAAV vector-based gene therapy constructs described herein and provide recombinant vectors, virus particles, infectious virions, and isolated host cells that comprise one or more of the rAAV nucleic acid vectors described herein.

Additionally, compositions and therapeutic and/or diagnostic kits comprise one or more of the disclosed AAV nucleic acid vector or AAV particle compositions, formulated with one or more additional immunosuppressive agents or prepared with one or more instructions for their use.

In one aspect, compositions comprise recombinant adeno-associated viral (rAAV) nucleic acid vectors, virions, viral particles, and pharmaceutical formulations thereof, useful for delivering genetic material encoding one or more beneficial or therapeutic products to mammalian cells and tissues. In some embodiments, the compositions and methods treat, prevent, and ameliorate the symptoms of one or more mammalian inflammatory diseases, including autoimmune diseases such as multiple sclerosis (MS) and the like.

In some embodiments, rAAV-based expression constructs encode one or more mammalian therapeutic agent(s) (including, but not limited to, for example, protein(s), polypeptide(s), peptide(s), enzyme(s), antibodies, antigen-binding fragments, variants, and/or active fragments thereof), for use in the treatment, prophylaxis, and/or amelioration of one or more symptoms of a mammalian disease, dysfunction, injury, and/or disorder.

The improved nucleic acid vectors and expression systems may also optionally further comprise a polynucleotide that comprises one or more polylinkers, restriction sites, and/or multiple cloning region(s) to aid insertion (cloning) of one or more selected genetic elements, genes of interest, or therapeutic or diagnostic constructs into the rAAV vector at a selected site within the vector. In further embodiments, the exogenous polynucleotide(s) that may be delivered into suitable host cells by the rAAV nucleic acid vectors disclosed herein are of bacterial and/or mammalian origin, with polynucleotides encoding one or more polypeptides or peptides of human, non-human primate, porcine, bovine, ovine, feline, canine, equine, caprine, or lupine origin.

The exogenous polynucleotide that may be delivered into host cells by the disclosed viral nucleic acid vectors, in certain embodiments, encodes one or more proteins, one or more polypeptides, one or more peptides, one or more enzymes, or one or more antibodies (or antigen-binding fragments thereof), or may express one or more siRNAs, gRNA, ribozymes, antisense oligonucleotides, PNA molecules, or any combination thereof. When combinational gene therapies are desired, two or more different molecules may be produced from a single rAAV expression system, or a selected host cell may be transfected with two or more unique rAAV expression systems, each of which may comprise one or more distinct polynucleotides that encode a therapeutic agent.

In other embodiments, rAAV nucleic acid vectors are contained within an infectious adeno-associated viral particle, virion, or pluralities of such virions or infectious particles. Such vectors, particles, and virions may be contained within one or more diluents, buffers, physiological solutions, or pharmaceutical vehicles or formulated for administration to a mammal in one or more diagnostic, therapeutic, and/or prophylactic regimens.

The disclosure also concerns host cells that comprise at least one disclosed rAAV nucleic acid expression vectors or one or more virus particles or virions that comprise such an expression vector.

Compositions comprising one or more of the disclosed rAAV nucleic acid vectors, expression systems, infectious rAAV particles, or host cells also form part disclosure, and particularly those compositions that further comprise at least a first pharmaceutically-acceptable excipient for use in therapy and for use in manufacturing medicaments for treating one or more mammalian inflammatory diseases, monogenic diseases, disorders, dysfunctions, or trauma. Such pharmaceutical compositions may optionally further comprise one or more diluents, buffers, liposomes, a lipid, a lipid complex. Alternatively, the rAAV nucleic acid vectors or rAAV particles disclosure may be comprised within a plurality of microspheres, nanoparticles, liposomes, or any combination thereof.

The present disclosure also provides kits comprising one or more of the disclosed rAAV nucleic acid vectors (as well as one or more virions, viral particles, transformed host cells, or pharmaceutical compositions comprising such vectors, virions, particle, or host cells); and instructions for using such kits in one or more therapeutic, diagnostic, and/or prophylactic clinical embodiments. Such kits may further comprise one or more reagents, restriction enzymes, peptides, therapeutics, pharmaceutical compounds, or means for delivery of the composition(s) to host cells, or to an animal (e.g., syringes, injectables, and the like), in one or two or multiple doses and formulated with one or more additional immunosuppressive agents.

Exemplary kits comprise those for treating, preventing, or ameliorating the symptoms of a disease, deficiency, dysfunction, and/or injury, or may include components for the large-scale production of the viral vectors themselves, such as for commercial sale or use by others, including, e.g., virologists, medical professionals, and the like.

The present disclosure provides methods of use of the disclosed rAAV nucleic acid vectors, virions, expression systems, compositions, and host cells to prepare medicaments for diagnosing, preventing, treating, or ameliorating at least one or more symptoms of a disease, a dysfunction, a disorder, an abnormal condition, a deficiency, injury, or trauma in an animal, and in particular, one or more monogenic or autoimmune diseases in humans.

Compositions comprising one or more of the disclosed rAAV nucleic acid vectors, expression systems, infectious rAAV particles, and host cells also form part disclosure. In certain embodiments, compositions further comprise at least a first pharmaceutically acceptable excipient for use in manufacturing medicaments and methods involving therapeutic administration of such rAAV nucleic vectors, rAAV particles, and host cells.

The present disclosure also provides methods of use of the disclosed nucleic acid vectors, virions, expression systems, compositions, and host cells described herein to prepare medicaments for treating or ameliorating the symptoms of monogenic or autoimmune diseases in humans, such as MS or Duchenne muscular dystrophy (DMD), in combination with immunosuppressive agents to support one or two or multiple doses of an rAAV vector-based gene therapy construct.

In some embodiments of any one of the methods provided, the method further comprises administering an mTOR inhibitor, e.g., rapamycin (Sirolimus), hCTLA4Ig (Abatacept), anti-CD20 antibody (equivalent to rituximab for humans), IL-2 complex, prednisone, anti-CD52 antibody (equivalent to alemtuzumab for humans), anti-CD19 antibody, anti-CD79 antibody, ibrutinib, mycophenolate mofetil, Dimethyl fumarate (Tecfidera), and vamorolone alone or in combination to support one or two or multiple doses of an rAAV vector-based gene therapy construct.

In certain embodiments, the method further comprises administering a complement inhibitor. Examples of suitable complement inhibitors include, but are not limited to, eculizumab (Soliris), human C1-esterase inhibitor (Berinert, and Cinryze), OMS721, MASP2 inhibitor (Omeros), Amy 101 (Amyndas), APL2, a C3 targeting peptide (Apellis), ACH-4471, a Factor D binding antagonist (Achillion), LNP023, a Factor B blocking compound (Novartis), and the C5a receptor 1 targeting Avacopan (Chemocentryx).

Human C1-esterase inhibitor is a C1 inhibitor indicated for prophylaxis and treatment of Hereditary Angioedema (HAE), a human genetic disorder caused by a shortage of C1 inhibitor activity in an overreaction of the immune system. It comprises purified endogenous complement component-1 esterase inhibitor (hC1INH) isolated from human plasma. The primary function of endogenous C1INH is to regulate the activation of the complement and contact system pathways.

OMS 721 binds to the lectin pathway protease MASP2. C3 targeting proteins include APL2 (Apellis) and AMY 101 (Amyndas). ACH-4771 is a small Factor D inhibitor that is applied orally and blocks the catalytic side of Factor D, a protease that cleaves in its active state Factor B. Inactive Factor D, the alternative pathway convertase C3bBb is not formed, and complement activation does not proceed. The other orally administered inhibitor, LPN023, binds to Factor B's active site, inhibiting the alternative pathway C3 convertase and blocks C3 cleavage. Thus, different inhibitors are currently evaluated, targeting different levels of the complement cascade, the activation level, the lectin pathway, the C3 convertase of the AP, C3- and C5.

Several compounds target complement at the level of C5. Eculizumab and the new version ravulizumab (both Alexion) bind to C5 and block activation of the protein. Eculizumab is an immunoglobulin G-kappa (IgGκ) consisting of human constant regions and murine complementarity-determining regions grafted onto human framework light and heavy chain variable regions. The compound contains two 448-amino acid heavy chains and two 214-amino acid light chains and has a molecular weight of approximately 148 kilodaltons (kDa). Coversin is a tick-derived C5 binding protein (Akari) and C5 inhibitor. Cemdisiran blocks C5 synthesis as an RNAi targeted strategy (Alnylam) and LFG-316 (Novartis). The complement inflammatory C5a-C5aR1 axis is inhibited by IFX-1 (InflaRx) and Avacopan (Chemocentryx).

In some embodiments, an rAAV nucleic acid vector described herein comprises inverted terminal repeat sequences (ITRs), such as those derived from a wild-type AAV genome, such as the AAV2 genome. In some embodiments, the rAAV nucleic acid vector further comprises a nucleic acid segment that comprises a transgene (also referred to as a heterologous nucleic acid molecule) operably linked to a promoter and, optionally, other regulatory elements, wherein the ITRs flank the nucleic acid segment.

In some embodiments, the promoter is a mammalian cell-specific or a mammalian tissue-specific promoter.

In some embodiments, the rAAV nucleic acid vector is encapsulated by an rAAV particle as described herein. The rAAV particle may be of any AAV serotype (e.g., 1, 2, 3, 4, 5, 6, 7, 8, 9, or 10), including any derivative (including non-naturally occurring variants of a serotype) or pseudotype. In some embodiments, the rAAV particle is an AAV8 particle, which may be pseudotyped with AAV2 ITRs. Non-limiting examples of derivatives and pseudotypes include AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAEl/2, AAV clone 32/83, AAVShH10, AAV2 (Y->F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A I, Schaffer D V, Samulski R J.). In some embodiments, the rAAV particle is a pseudotyped rAAV particle, which comprises (a) a nucleic acid vector comprising ITRs from one serotype (e.g., AAV2) and (b) a capsid comprised of capsid proteins derived from another serotype (e.g., AAV1, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Exemplary rAAV nucleic acid vectors include single-stranded (ss) or self-complementary (sc) AAV nucleic acid vectors, such as single-stranded or self-complementary recombinant viral genomes.

Methods of producing rAAV particles and nucleic acid vectors are also known in the art and commercially available (see, e.g., Zolotukhin et al., “Production and purification of serotype 1, 2, and 5 recombinant adeno-associated viral vectors.” Methods 28 (2002) 158-167; US 2007/0015238, and US 2012/0322861, which are incorporated herein by reference; and plasmids and kits available from ATCC and Cell Biolabs, Inc.). For example, a plasmid containing the nucleic acid vector sequence may be combined with one or more helper plasmids, e.g., that contain a rep gene (e.g., encoding Rep78, Rep68, Rep52, and Rep40) and a cap gene (encoding VP1, VP2, and VP3, including a modified VP3 region as described herein), and transfected into a producer cell line such that the rAAV particle can be packaged and subsequently purified.

In some embodiments, the one or more helper plasmids include a first helper plasmid comprising a rep gene and a cap gene and a second helper plasmid comprising a E1a gene, a E1b gene, a E4 gene, a E2a gene, and a VA gene. In some embodiments, the rep gene is a rep gene derived from AAV2, and the cap gene is derived from AAV2 and includes modifications to the gene to produce a modified capsid protein described herein.

Helper plasmids, and methods of making such plasmids, are known in the art and commercially available (see, e.g., pDM, pDG, pDPlrs, pDP2rs, pDP3rs, pDP4rs, pDP5rs, pDP6rs, pDG(R484E/R585E), and pDP8.ape plasmids from PlasmidFactory, Bielefeld, Germany. Other products and services available from Vector Biolabs, Philadelphia, Pa., Cellbiolabs, San Diego, Calif., Agilent Technologies, Santa Clara, Calif., and Addgene, Cambridge, Mass. See Grimm et al. (1998), “Novel Tools for Production and Purification of Recombinant Adenoassociated Virus Vectors,” Human Gene Therapy, Vol. 9, 2745-2760; Kem, A. et al. (2003), “Identification of a Heparin-Binding Motif on Adena-Associated Virus Type 2 Capsids,” Journal of Virology, Vol. 77, 11072-11081.; Grimm et al. (2003), “Helper Virus-Free, Optically Controllable, and Two-Plasmid-Based Production of Adena-associated Virus Vectors of Serotypes 1 to 6,” Molecular Therapy, Vol. 7, 839-850; Kronenberg et al. (2005), “A Conformational Change in the Adena-Associated Virus Type 2 Capsid Leads to the Exposure of Hidden VP1 N Termini”, Journal of Virology, Vol. 79, 5296-5303; and Moullier, P. and Snyder, R. O. (2008), “International efforts for recombinant adeno-associated viral vector reference standards,”Molecular Therapy, Vol. 16, 1185-1188.

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO₄-mediated transfection, lipids, or polymeric molecules such as polyethyleneimine (PEI) with the helper plasmid and a plasmid containing a nucleic acid vector described herein. The HEK293 cells are then incubated for at least 60 hours to allow for rAAV particle production. Alternatively, Sf9-based producer stable cell lines are infected with a single recombinant baculovirus containing the nucleic acid vector. In another example, HEK293 or BHK cell lines are infected with an HSY containing the nucleic acid vector and optionally one or more helper HSYs containing rep and cap ORFs as described herein and the adenoviral VA, E2A (DBP), and E4 genes under the transcriptional control of their native promoters.

The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

### Guide RNA (For CRISPR/Cas Endonucleases)

A nucleic acid molecule that binds to a class 2 CRISPR/Cas endonuclease (e.g., a Cas9 protein; a type V or type VI CRISPR/Cas protein; a Cpf 1 protein; etc.) and targets the complex to a specific location within a target nucleic acid is a “guide RNA” or “CRISPR/Cas guide nucleic acid” or “CRISPR/Cas guide RNA.”

A guide RNA provides target specificity to the complex (the RNP complex) by comprising a targeting segment, which comprises a guide sequence (a “targeting sequence”), a nucleotide sequence complementary to a sequence of a target nucleic acid. A guide RNA can be referred to by the protein to which it corresponds. For example, when the class 2 CRISPR/Cas endonuclease is a Cas9 protein, the corresponding guide RNA can be referred to as a “Cas9 guide RNA.” Likewise, as another example, when the class 2CRISPR/Cas endonuclease is a Cpf 1 protein, the corresponding guide RNA is a “Cpf1 guide RNA.”

In some embodiments, a guide RNA includes two separate nucleic acid molecules: An “activator” and a “targeter” and is referred to herein as a “dual guide RNA,” a “double-molecule guide RNA,” a “two-molecule guide RNA,” or a “dgRNA.” In some embodiments, the guide RNA is one molecule (e.g., for some class 2 CRISPR/Cas proteins, the corresponding guide RNA is a single molecule; and in some cases, an activator and targeter are covalently linked to one another, e.g., via intervening nucleotides). The guide RNA is referred to as a “single-guide RNA,” a “single-molecule guide RNA,” a “one -molecule guide RNA,” or simply “sgRNA.”

In some embodiments, a subject CRISPR/Cas guide RNA (e.g., a Cas9 guide RNA) targets a target sequence depicted in Table 2. In some embodiments, a subject CRISPR/Cas guide RNA (e.g., a Cas9 guide RNA) targets a target sequence depicted in Table 1.

Examples of (i) target sequences (non-complementary strand) of target DNA, and (ii) guide sequences of CRISPR/Cas guide RNAs (e.g., for CRISPR/Cas proteins such as 5 pyogenes Cas9 that have a PAM requirement of NGG in the non-complementary strand), where the first targeted sequence is within intron 44 of the human dystrophin gene and the second targeted sequence is within intron 55 of the human dystrophin gene. A guide sequence targeted to a target sequence within intron 44 of the human dystrophin gene is referred to as a “44” series guide sequence. A guide sequence targeted to a target sequence within intron 55 of the human dystrophin gene is referred to as a “55” series guide sequence.

For example, in some cases, a first CRISPR/Cas guide RNA (e.g., a Cas9 guide RNA) comprises a guide sequence that comprises a sequence SEQ ID NO:3 (which sequences are 20 nucleotides long and hybridize to a target sequence within intron 44 of the human dystrophin gene).

In some cases, a second CRISPR/Cas guide RNA (e.g., a Cas9 guide RNA) comprises a guide sequence that comprises sequence SEQ ID NO:7 (which sequences are 20 nucleotides long and hybridize to a target sequence within intron 55 of the human dystrophin gene).

TABLE 1
guide sequences of guide RNAs and non-complementary strands of target
sequences.

| | | | | | Followed by | SEQ ID |
|---|---|---|---|---|---|---|
| Intron | Site | | Length | Sequence | PAM | NO: |
| 44 | 4 | Non- | 20 nt | GTTGAAATTAAACT | TGG | 1 |
| | (44C4) | complementary | | ACACAC | | |
| | | strand of target | 17 nt | GAAATTAAACTAC | TGG | 2 |
| | | sequence | | ACAC | | |
| | | Guide sequence | 20 nt | GUUGAAAUUAAAC | | 3 |
| | | of Guide RNA | | UACACAC | | |
| | | | 17 nt | GAAAUUAAACUAC | | 4 |
| | | | | ACAC | | |
| 55 | 3 | Non- | 20 nt | TGTATGATGCTATA | AAG | 5 |
| | (55C3) | complementary | | ATACCA | | |
| | | strand of target | 17 nt | ATGATGCTATAATA | AAG | 6 |
| | | sequence | | CCA | | |
| | | Guide sequence | 20 nt | UGUAUGAUGCUAU | | 7 |
| | | of Guide RNA | | AAUACCA | | |
| | | | 17 nt | AUGAUGCUAUAAU | | 8 |
| | | | | ACCA | | |

As used herein, the terms “engineered” and “recombinant” cells refer to a cell into which an exogenous polynucleotide segment has been introduced. The segment may be a DNA segment that leads to the transcription of a biologically active molecule. Therefore, engineered cells are distinguishable from naturally occurring cells, which do not contain a recombinantly introduced exogenous DNA segment. Engineered cells are, therefore, cells that comprise at least one or more heterologous polynucleotide segments introduced through the hand of man. To express a therapeutic agent per the present disclosure, one may prepare a tyrosine capsid-modified rAAV particle containing an expression vector that comprises a therapeutic agent-encoding nucleic acid segment under the control of one or more promoters. To bring a sequence “under the control of a promoter,” one positions the 5′ end of the transcription initiation site of the transcriptional reading frame, generally between about 1 and about 50 nucleotides “downstream” of (i.e., 3′ of) the chosen promoter. The “upstream” promoter stimulates transcription of the DNA and promotes the expression of the encoded polypeptide. This is the meaning of “recombinant expression” in this context.

Particular recombinant nucleic acid vector constructs comprise an rAAV nucleic acid vector containing a therapeutic gene of interest operably linked to one or more promoters capable of expressing the gene in one or more selected mammalian cells. Such nucleic acid vectors are described in detail herein. The genetic constructs disclosure may be prepared in various compositions and may also be formulated in appropriate pharmaceutical vehicles to administer to human or animal subjects. The rAAV molecules' disclosed and the compositions comprising them provide new and useful therapeutics to treat, control, and ameliorate symptoms of various disorders, diseases, injuries, and/or dysfunctions of the mammalian nervous system, in particular the treatment or amelioration of muscular dystrophy.

In some embodiments, the number of rAAV particles administered to a subject may range from 10⁶ to 10¹⁴ particles/ml or 10³ to 10¹⁵ particles/ml, or any values therebetween for either range, for example, about 10⁶, 10⁷, 10⁸, 10⁹, 10¹⁰, 10¹¹, 10¹², 10¹³, or 10¹⁴ particles/ml. In one embodiment, rAAV particles of higher than 10¹³ particles/mL may be administered. In some embodiments, the number of rAAV particles administered to a subject may range from 10⁶ to 10¹⁴ vector genomes/mL (vgs/mL) or 10³ to 10¹⁵ vgs/ml, or any values therebetween, such as about 10⁶, 10⁷, 10⁸, 10⁹, 10¹⁰, 10¹¹, 10¹², 10¹³, or 10¹⁴ vgs/ml. In one embodiment, rAAV particles of higher than 10¹³ vgs/ml are administered.

The rAAV particles can be administered as a single dose or divided into two or more administrations to achieve therapy of the particular disease or disorder being treated. In some embodiments, 0.0001 mL to 10 mLs, e.g., 0.001 mL, 0.01 mL, 0.1 mL, 1 mL, 2 mL, 5 mL or 10 mL, are delivered to a subject. In some embodiments, the number of rAAV particles administered to a subject may range from 10⁶-10¹⁴ vg/kg, or any values there in between, for example, about 10⁶, 10⁷, 10⁸, 10⁹, 10¹⁰, 10¹¹, 10¹², 10¹³, or 10¹⁴ vgs/kg.

In some embodiments, the disclosure provides formulations of one or more viral-based compositions disclosed herein in pharmaceutically acceptable solutions for administration to a cell or an animal, alone or in combination, with one or more other modalities of therapy, and in particular, for therapy of human cells, tissues, and diseases affecting man, to support one or two or multiple doses of an rAAV vector-based gene therapy construct.

If desired, rAAV particles described herein may be administered in combination with other agents as well, such as, e.g., proteins or polypeptides or various pharmaceutically active agents, such as immunosuppressive agents, including one or more systemic or topical administrations of therapeutic polypeptides, biologically active fragments, or variants thereof to support one or two or multiple doses of an rAAV vector-based gene therapy construct. The rAAV particles may be delivered with various other agents. Such compositions may be purified from host cells or other biological sources or may be chemically synthesized as described herein.

Typically, these formulations may contain at least about 0.1% of the therapeutic agent (e.g., rAAV particle) or more. However, the percentage of the active ingredient(s) may, of course, be varied and may conveniently be between about 1 or 2% and about 70% or 80% or more of the weight or volume of the total formulation. Naturally, the amount of therapeutic agent(s) in each therapeutically useful composition may be prepared so that a suitable dosage will be obtained in any given unit dose of the compound. Factors such as solubility, bioavailability, biological half-life, route of administration, product shelf life, and other pharmacological considerations will be contemplated by one skilled in preparing such pharmaceutical formulations. As such, a variety of dosages and treatment regimens may be desirable.

In certain circumstances, it will be desirable to deliver rAAV particles in suitably formulated pharmaceutical compositions disclosed herein either subcutaneously, intraocularly, intravitreally, parenterally, intravenously, intracerebro-ventricularly, intramuscularly, intrathecally, orally, intraperitoneally, by oral or nasal inhalation, or by direct injection to one or more cells, tissues, or organs by direct injection. The pharmaceutical forms of the injectable compositions comprise sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as a lecithin, by the maintenance of the particle size, in the case of dispersion, and by the use of surfactants.

The term “carrier” refers to a diluent, adjuvant, excipient, or vehicle with which the rAAV particle is administered. Such pharmaceutical carriers can be sterile liquids, such as water and oils, including petroleum oil such as mineral oil, vegetable oil such as peanut oil, soybean oil, sesame oil, animal oil, or oil of synthetic origin. Saline solutions and aqueous dextrose and glycerol solutions can also be employed as liquid carriers. Other exemplary carriers include phosphate-buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose.

The compositions of the present disclosure can be administered to the subject being treated by standard routes including, but not limited to, pulmonary, intranasal, oral, inhalation, parenteral such as intravenous, topical, transdermal, intradermal, transmucosal, intraperitoneal, intramuscular, intracapsular, intraorbital, intravitreal, intracardiac, transtracheal, subcutaneous, subcuticular, intraarticular, subcapsular, subarachnoid, intraspinal, epidural, and intrastemal injection. In some embodiments, the composition is administered intravenously, by hepatic artery infusion, portal vein injection, or intrasplenic injection. In some embodiments, the composition comprises an AAV9 rAAV particle comprising an rAAV nucleic acid vector as described herein. The composition is administered intraperitoneally with one or more immunosuppressive agents chosen from rapamycin (Sirolimus), hCTLA4Ig (Abatacept), anti-CD20 antibody (the mouse equivalent of rituximab for humans), IL-2 complex, prednisone, anti-CD52 antibody (mouse equivalent to alemtuzumab for humans), anti-CD19 antibody, anti-CD79 antibody, ibrutinib, mycophenolate mofetil, dimethyl fumarate (Tecfidera), and vamorolone alone or in combination to support one or two or multiple doses of an rAAV vector-based gene therapy construct. In certain embodiments, the method further comprises administering a complement inhibitor.

To administer an injectable aqueous solution, for example, the solution may be suitably buffered. In certain embodiments, the liquid diluent is first rendered isotonic with sufficient saline or glucose. These aqueous solutions are especially suitable for intravenous, intramuscular, intravitreal, subcutaneous, and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed infusion site (see, for example, Remington's Pharmaceutical Sciences, 15th Edition, pages 1035-1038 and 1570-1580). Some variation in dosage may occur depending on the condition of the subject being treated. The person responsible for administration will, in any event, determine the appropriate dose for the individual subject. Moreover, for human administration, preparations should meet sterility, pyrogenicity, and the general safety and purity standards of, e.g., the FDA Office of Biologics standards.

Sterile injectable solutions may be prepared by incorporating the rAAV particles in the appropriate solvent with several other ingredients enumerated above, followed by filtered sterilization. Generally, dispersions are prepared by incorporating the various sterilized active ingredients into a sterile vehicle which contains the basic dispersion medium and the other ingredients from those enumerated above. In sterile powders for the preparation of sterile injectable solutions, exemplary methods of preparation are vacuum drying and freeze-drying techniques that yield a powder of the active ingredient plus any additional desired ingredient from a previously sterile-filtered solution thereof. Sterile injectable solutions may be prepared by incorporating the rAAV particles in the appropriate solvent with several of the other ingredients, which may be one or more immunosuppressive agents chosen from rapamycin (Sirolimus), hCTLA4Ig (Abatacept), anti-CD20 antibody (the mouse equivalent of rituximab for humans), IL-2 complex, prednisone, anti-CD52 antibody (mouse equivalent to alemtuzumab for humans), anti-CD19 antibody, anti-CD79 antibody, ibrutinib, mycophenolate mofetil, dimethyl fumarate (Tecfidera), and vamorolone alone or in combination to support one or two or multiple doses of an rAAV vector-based gene therapy construct, followed by filtered sterilization. In certain embodiments, the method further comprises administering a complement inhibitor.

The amount of rAAV particle compositions and time of administration of such compositions will be within the purview of the skilled artisan benefiting the present teachings. It is likely, however, that administering therapeutically effective amounts of the disclosed compositions may be achieved by a single administration, such as a single injection of sufficient numbers of viral particles to provide therapeutic benefit to the patient undergoing such treatment. Alternatively, in some circumstances, it may be desirable to provide multiple, or successive administrations of the compositions, either over a relatively short or a relatively prolonged period, as may be determined by the medical practitioner overseeing administering such compositions.

The composition may include rAAV particles or nucleic acid vectors either alone, or in combination with one or more additional active ingredients, which may be obtained from natural or recombinant sources or chemically synthesized. Per the present disclosure, polynucleotides, nucleic acid segments, nucleic acid sequences, and the like, include, but are not limited to, DNAs (including and not limited to genomic or extra-genomic DNAs), genes, peptide nucleic acids (PNAs), RNAs (including, but not limited to, rRNAs, mRNAs, and tRNAs), nucleosides, and suitable nucleic acid segments either obtained from natural sources, chemically synthesized, modified, or otherwise prepared or synthesized in whole or in part by the hand of man. Unless defined otherwise, all technical and scientific terms used herein have the same meaning as commonly understood by one of ordinary skill in the art to which this

Although any methods and compositions similar or equivalent to those described herein can be used in the practice or testing, the methods and compositions are described herein. For purposes, the following terms are defined below.

The term “subject,” as used herein, describes an organism, including mammals such as primates, to which treatment with the compositions according to the present invention can be provided. Mammalian species that can benefit from the disclosed treatment methods include, but are not limited to, humans, apes, chimpanzees, orangutans, monkeys, domesticated animals such as dogs and cats, and livestock such as horses, cattle, pigs, sheep, goats, mice, and chickens. In certain embodiments, the subject is a patient, such as a human being in need of treatment. An “identical patient” or “identical subject” means that treatment is administered to the same patient or the same subject.

In some embodiments, the patient has, is suspected of having, is at risk for developing, or has been diagnosed with Duchenne muscular dystrophy (DMD), Becker Muscular dystrophy (BMD, a mild form of DMD); an intermediate clinical presentation between DMD and BMD; and DMD-associated dilated cardiomyopathy (heart disease).

The term “treatment” or any grammatical variation thereof (e.g., treat, treating, and treatment etc.), as used herein, includes but is not limited to alleviating a symptom of a disease or condition; and/or reducing, suppressing, inhibiting, lessening, ameliorating, or affecting the progression, severity, and/or scope of a disease or condition.

The term “effective amount,” as used herein, refers to an amount capable of treating or ameliorating a disease or condition or otherwise capable of producing an intended therapeutic effect.

The term “promoter,” as used herein, refers to a region or regions of a nucleic acid sequence that regulates transcription.

The term “regulatory element,” as used herein, refers to a region or regions of a nucleic acid sequence regulating transcription. Exemplary regulatory elements include, but are not limited to, enhancers, post-transcriptional elements, transcriptional control sequences, and such like.

The term “vector,” as used herein, refers to a nucleic acid molecule (typically comprised of DNA) capable of replication in a host cell and/or to which another nucleic acid segment can be operatively linked to bringing about replication of the attached segment. A plasmid, cosmid, or virus is an exemplary vector.

The term “substantially corresponds to,” “substantially homologous,” or “substantial identity,” as used herein, denote a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84, or even 85 percent sequence identity, and such as at least about 86, 87, 88, 89, 90, 91, 92, 93, 94, or 95 percent sequence identity. In certain embodiments, highly-homologous sequences often share greater than at least about 96, 97, 98, or 99 percent sequence identity between the selected sequence and the reference sequence to which it was compared.

The percentage of sequence identity may be calculated over the entire length of the sequences to be compared or calculated by excluding small deletions or additions that total less than about 25 percent or so of the chosen reference sequence. The reference sequence may be a subset of a larger sequence, such as a portion of a gene or flanking sequence or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides.

When highly homologous fragments are desired, the extent of percent identity between the two sequences will be at least about 80%, such as at least about 85%, for example, about 90% or 95% or higher, as readily determined by one or more of the sequence comparison algorithms well-known to those of skill in the art, such as, e.g., the PASTA program analysis described by Pearson and Lipman (1988).

The term “operably linked,” as used herein, refers to that the nucleic acid sequences being linked are typically contiguous or substantially contiguous and, where necessary, to join two protein-coding regions, contiguous and in reading frame. However, since enhancers generally function when separated from the promoter by several kilobases and intronic sequences may be variable lengths, some polynucleotide elements may be operably linked but not contiguous.

The term “biologically active,” as used herein, refers to a variant nucleic acid or protein sequence with substantially the same activity.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1

### Efficacy of Multiple AAV9 Injections in Combination with Immunosuppressive (IS) Drug Protocol

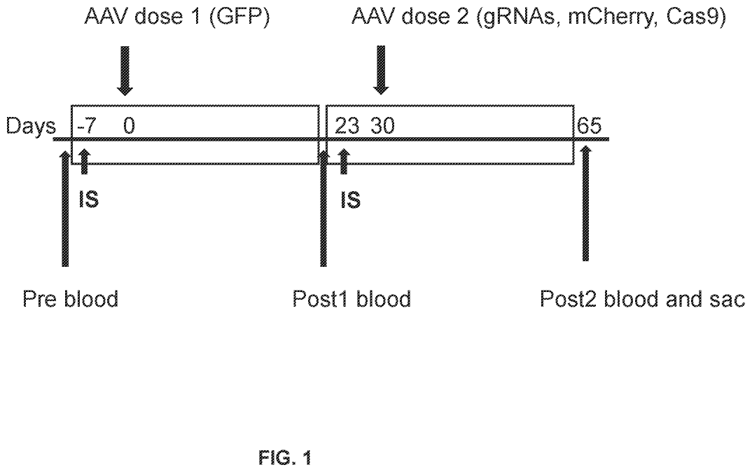

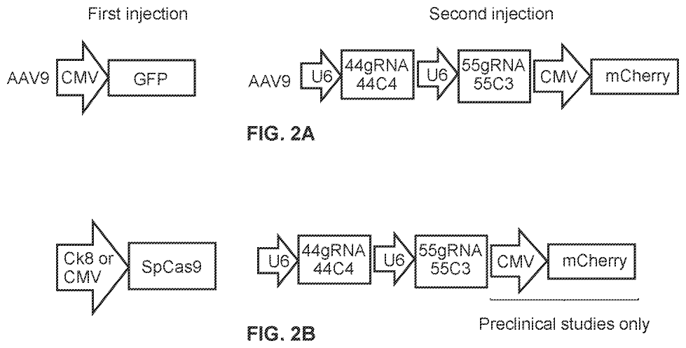

Experimental overview. Different immunosuppressive drug regimens were assessed for their ability to support multiple injections of AAV9 to be efficacious. The experimental paradigm is outlined in FIG. 1. Mice were given an immunosuppression protocol (IS) commencing at Day-7 (1^{st} dose—AAV injection) and re-dosed (2^{nd} dose—AAV9 injection) approximately 4 weeks after the first dose. The 1^{st} dose consisted of AAV9-CMV-GFP at a concentration of 1.16×10¹⁴ vector genomes (vgs)/kg (˜3×10¹² vgs/mouse). In some cases, the 1^{st} dose also included AAV9-Ck8-Cas9. The 2^{nd} dose consisted of AAV9-target (containing mCherry and 45-55 gRNAs21) at a concentration of 1.16×10¹⁴ vgs/kg and AAV9-Ck8-Cas9 at a concentration of 1.16×10¹⁴ vgs/kg each vector (FIGS. 2A and 2B). Immunomodulation protocols were tested to determine the efficacy of repeat dosing by GFP and mCherry expression.

Immunosuppressive drugs. The following combinations were tested: 1) prednisone alone; 2) anti-CD20 antibody and sirolimus; 3) anti-CD20 antibody, sirolimus, and prednisone; 4) anti-CD20 antibody, sirolimus, prednisone, and CTLA4-Ig; 5) anti-CD20 antibody, sirolimus, prednisone, and IL-2 complex (IL-2C); 6) anti-CD20 antibody, prednisone, and CTLA4-Ig; 7) sirolimus, prednisone, and CTLA4Ig; 8) anti-CD20 antibody, sirolimus, prednisone, and anti-CS antibody; 9) anti-CD79b antibody, sirolimus, and prednisone; 10) anti-CD19 antibody, sirolimus, and prednisone; 11) ruxolitinib and prednisone; and 12) ibrutinib, sirolimus, and prednisone. Additional agents that will be tested are selected from an anti-CD52 antibody, mycophenolate mofetil, dimethyl fumarate, and vamorolone. The drugs and their dosing are listed in Table 2.

TABLE 2
Immunosuppressive drugs used in the protocol

| | Dose | | Duration |
|---|---|---|---|
| hCTLA4Ig | 10 | mg/kg i.p. | D 0, D 4, D 14, D 28 and/or |
| (Abatacept) | | | D 35, D 39, D 49, D 63 |
| Anti-CD20 antibody | 10 | mg/kg i.v. or r.o | Every week for 3 weeks around the |
| (mouse equivalent | | | time of AAV injection (e.g., D (−7), |
| of rituximab) | | | D 0, D 7 and D 23, D 30, D 37) |
| Sirolimus | 1 | mg/kg i.p. | Every day starting |
| (rapamycin) | | | at D (−7) |
| IL2 complex | 0.5 μg IL-2 preincubated | | 3x/week starting |
| | with 5 μg IL-2 antibody | | at D (−5) |
| | JES6-1A12 i.p. | | |
| Prednisone | 1 | mg/kg i.p. | Every day starting |
| | | | at D (−1) or D (−7) |
| Anti-C5 antibody | 40 | mg/kg i.p. | Day before AAV injection |
| (equivalent of | | | (e.g. D (−1), D 29) |
| eculizumab) | | | |
| Anti-CD79 or CD79b | 20 | mg/kg i.p. | Every week for 3 weeks around the |
| antibody | | | time of AAV injection (e.g., D (−7), |
| | | | D 0, D 7 and D 23, D 30, D 37) |
| Ibrutinib | 20 | mg/kg i.p. | Every day starting |
| | | | at D (−7) |
| Ruxolitinib | 45 | mg/kg i.p. | Every day starting |
| | | | at D (−7) |
| Anti-CD19 | 6 | mg/kg i.p. | Every 3 days for 2 weeks around |
| antibody | | | the time of AAV injection and |
| | | | then every 5 days after |

These immunosuppressive agents will be tested

| | | | |
|---|---|---|---|
| Anti-CD52 antibody | 2-10 | mg/kg i.p. | Every week for 3 weeks around the |
| (mouse equivalent | | | time of AAV injection (e.g., D (−7), |
| Alemtuzumab) | | | D 0, D 7 and D 23, D 30, D 37) |
| Mycophenolate | 60 | mg/kg i.p. | Every day starting |
| mofetil | | | at D (−7) |
| Dimethyl fumarate | 15 | mg/kg i.p. | Every day starting |
| (Tecfidera) | | | at D (−7) |

Intraperitoneal injection (i.p.),
intravenous injection (i.v.),
retro-orbital (r.o.)

Mice. Mdx or hDMD de145 mdx mice at 6-10 weeks of age were used. They were maintained according to UCLA Animal Research Committee approval. Mice were injected with immunosuppressive (IS) drugs intraperitoneally (i.p.) or via retro-orbital (r.o.) injection in PBS as outlined in Table 1. AAV9 was injected r.o. in HBSS (Hank's Balanced Salt Solution).

AAV. AAV was purchased from Virovek Inc. (Hayward, Calif.) or made in-house through triple transfection of plasmids in HEK293 AAV cells using TransIT-VirusGEN (Mirus Bio, Madison Wis.) and purified by iodixanol gradient ultracentrifugation or by Virovek Inc.

Muscle assessment. After the experiments, various muscles and organs were harvested, including heart, diaphragm, tibialis anterior, soleus, triceps, gastrocnemius, quadriceps, liver, spleen, and kidney. For muscle tissue, portions were fixed in PFA for immunostaining, frozen in OCT, and frozen in liquid nitrogen for Western blotting.

Blood. Blood draws were taken through retro-orbital bleeding or the tail vein at indicated time points, and plasma was isolated by centrifugation and stored at −80° C., and PBMCs recovered by a Ficoll-Paque gradient (GE Healthcare, Chicago, Ill.) for cryopreservation.

Staining. Ten-micron cryosections were obtained throughout the muscles. Autofluorescence was quenched using TrueBlack Lipofuscin (Biotium, Fremont Calif.). The samples were blocked in 5% horse serum and 10% goat serum and stained with anti-laminin primary antibody at 1:200 (L9393, Sigma-Aldrich, St Louis, Mo.) overnight. The following day a rabbit Alex Fluor 647 secondary antibody (Thermo Fisher Invitrogen, Carlsbad Calif.) was applied. Laminin staining and endogenous GFP and mCherry signal were imaged.

Western blotting. Muscle tissue was solubilized in reducing sample buffer (50 mm Tris-HCl, pH 6.8, 10% glycerol, 2% sodium dodecyl sulfate (SDS), and 100 mm (3-mercaptoethanol) with 1×Halt™ protease and phosphatase inhibitors (Thermo Fisher Scientific, Waltham Mass.). Protein samples were resolved on 12% tris-glycine gels by SDS-PAGE and then transferred to nitrocellulose membrane (Millipore, Burlington Mass.). Membranes were blocked for 1 hour in 4% nonfat dry milk in TBS with 0.1% Tween 20 and incubated in primary antibodies to GFP and mCherry diluted in 4% BSA. Horseradish peroxidase-conjugated anti-rabbit IgG and anti-mouse IgG secondary antibodies were used at 1:2,000 dilutions in 4% BSA. Immunoblots were developed using enhanced chemiluminescence (Radiance ECL; Azure Biosystems, Dublin Calif.).

ELISA. Anti-AAV9 antibodies were measured using an ELISA with purified AAV9 vector as antigens as described. In short, 8×10⁸ vg/50 μl/well AAV9 vector in coating buffer along with a corresponding standard curve of mouse IgG2a was applied to plates and blocked. Diluted serum samples were added, incubated, and then visualized with anti-mouse IgG2a horseradish peroxidase reaction with 3,3′,5,5′ tetramethylbenzidine (TMB, Thermo Fisher Invitrogen, Carlsbad Calif.) by 650 nm absorbance reading. ELISAs for Cas9 and dystrophin will be done similarly.

### Example 2

### mCherry Expression After Immunosuppressive Administration

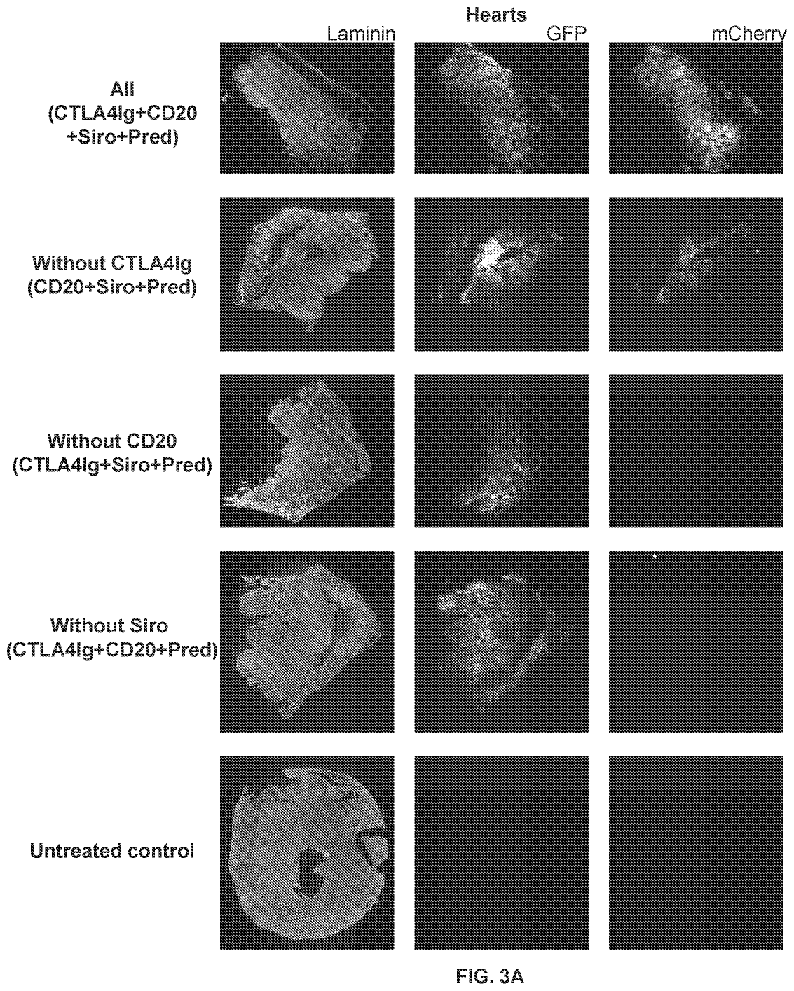

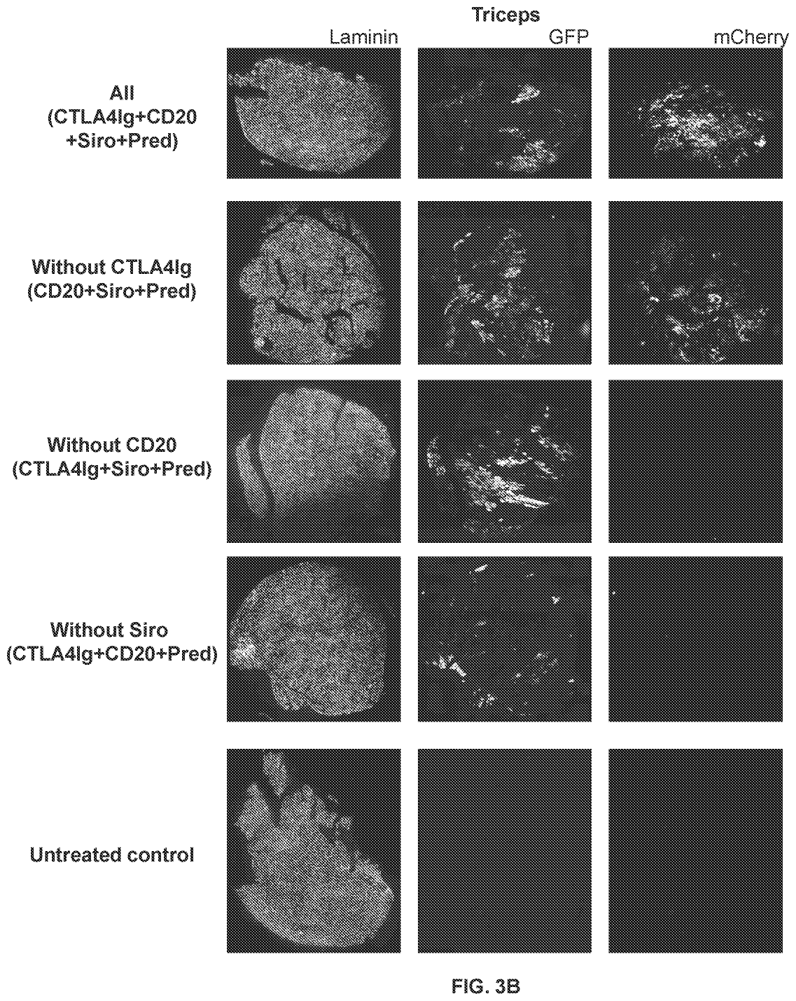

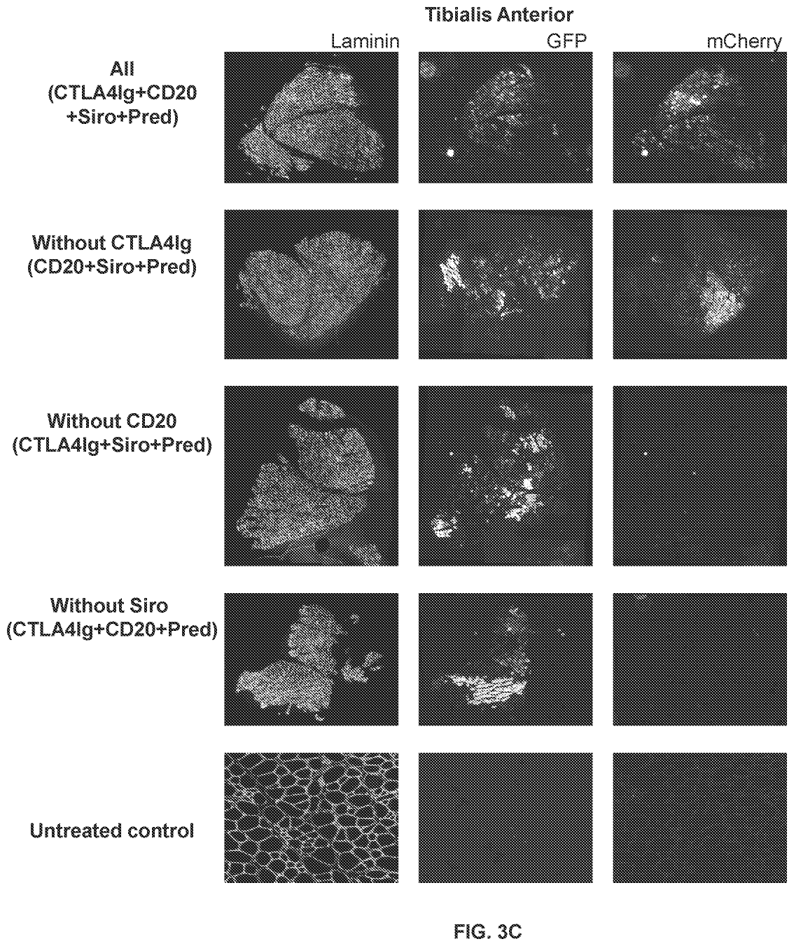

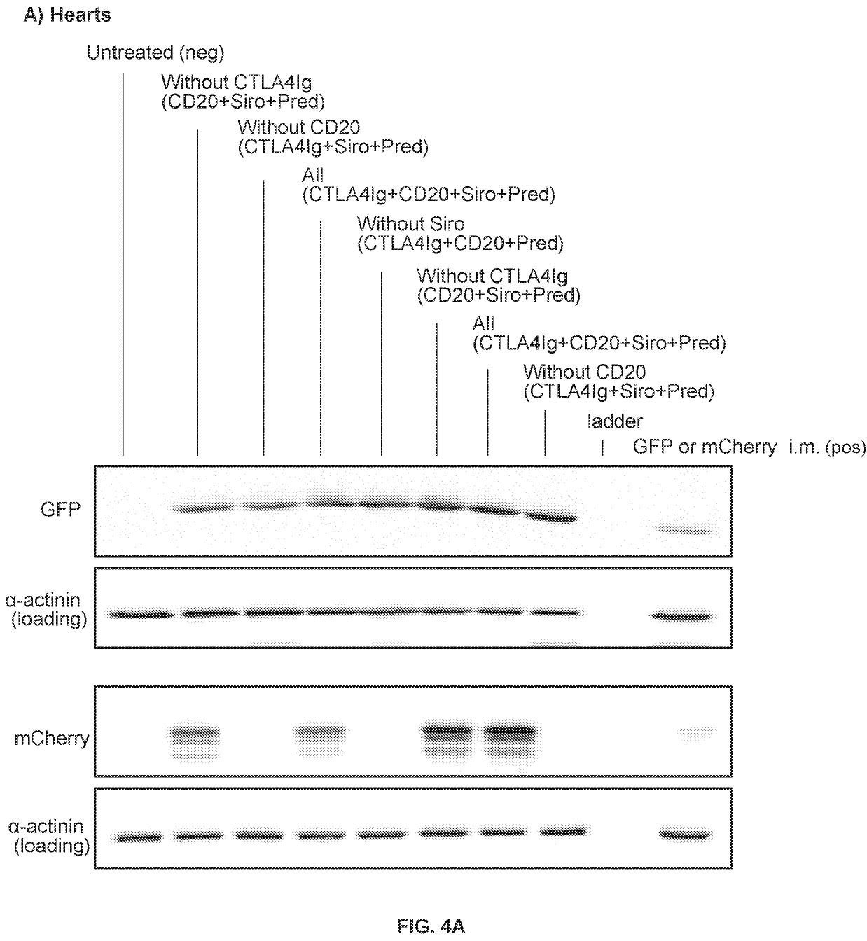

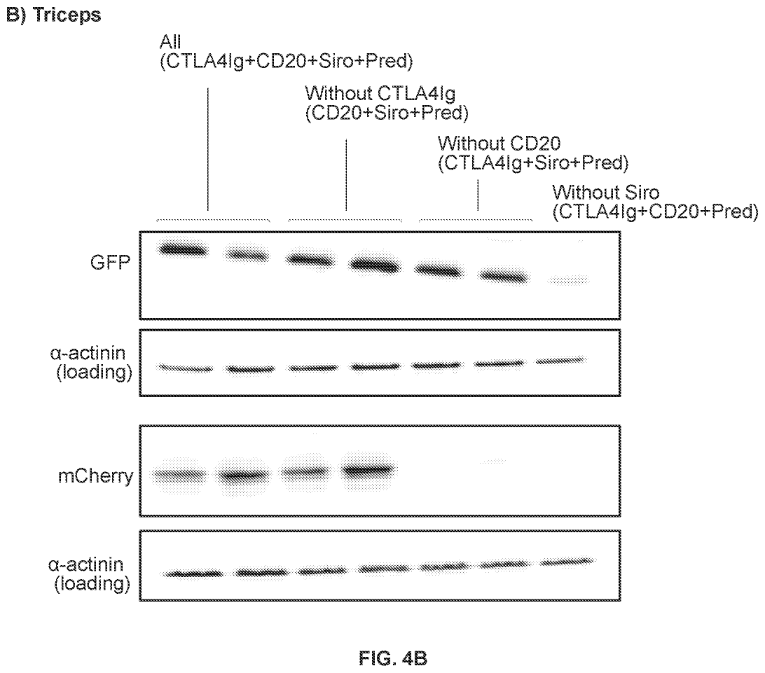

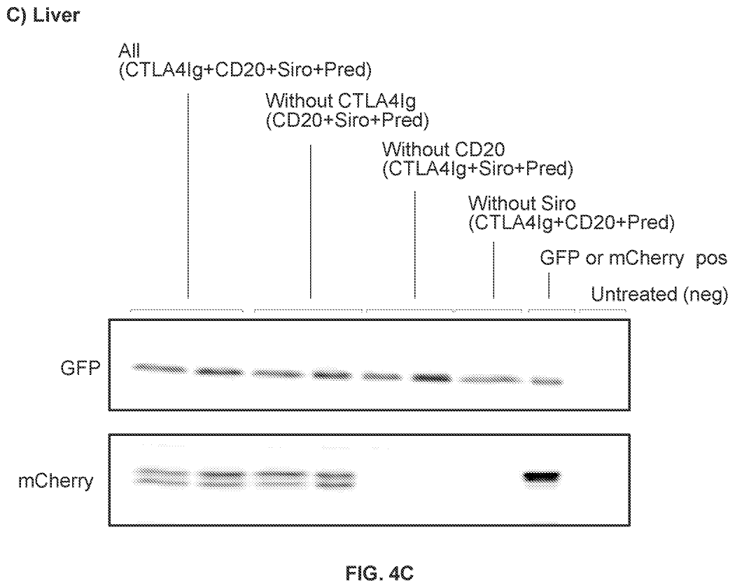

Redosing was assessed by looking for mCherry expression, which was only present in the second injection. By assessing which IS protocols did not allow for mCherry expression, some IS drugs critical for redosing AAV were determined. In one example, anti-CD20 antibody and sirolimus were discovered to be essential for redosing. When each component was removed individually, the expression of the second AAV injection (mCherry) was rejected and not detectable in the muscles (FIG. 3 and FIG. 4). IS regimens consisting of 1) anti-CD20 antibody, sirolimus, and prednisone, and 2) anti-CD20 antibody, sirolimus, prednisone, and CTLA4-Ig did allow for redosing, with similar levels of GFP and mCherry detectable across multiple muscles, including heart, triceps, and tibialis anterior, by immunostaining and Western blotting (FIG. 3 and FIG. 4). A similar trend was also observed in the liver (FIG. 4).

### Example 3

### Redosing (Assessment of mCherry Expression) After Immunosuppressive Regimen Administration with AAV

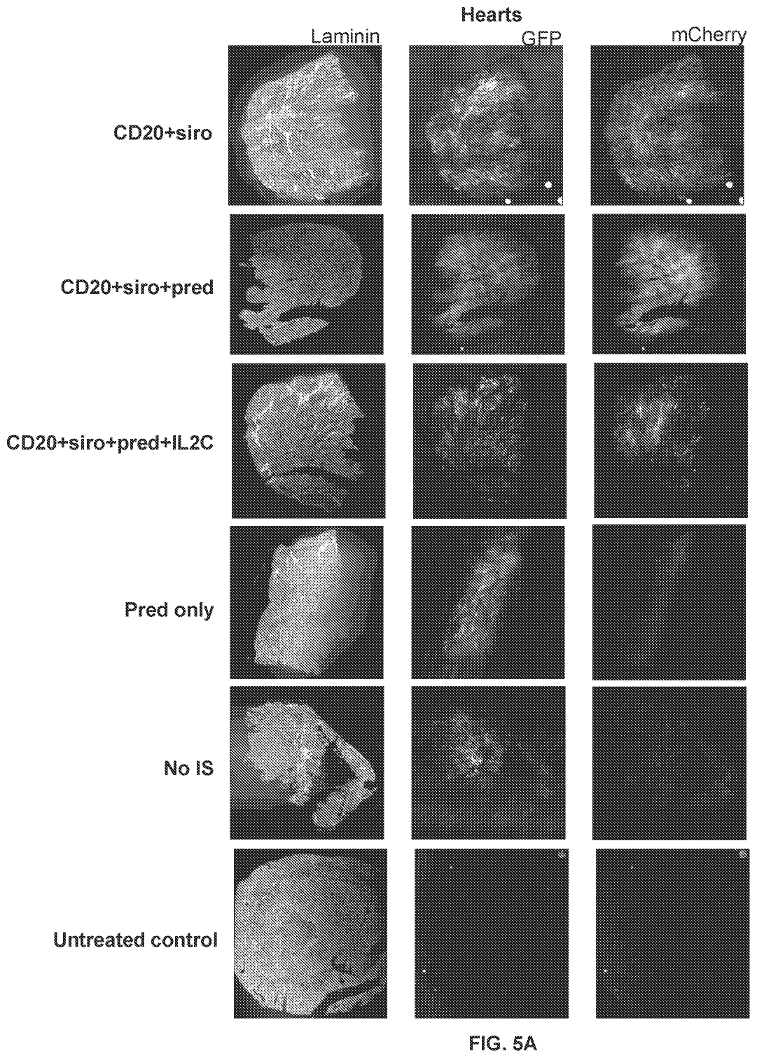

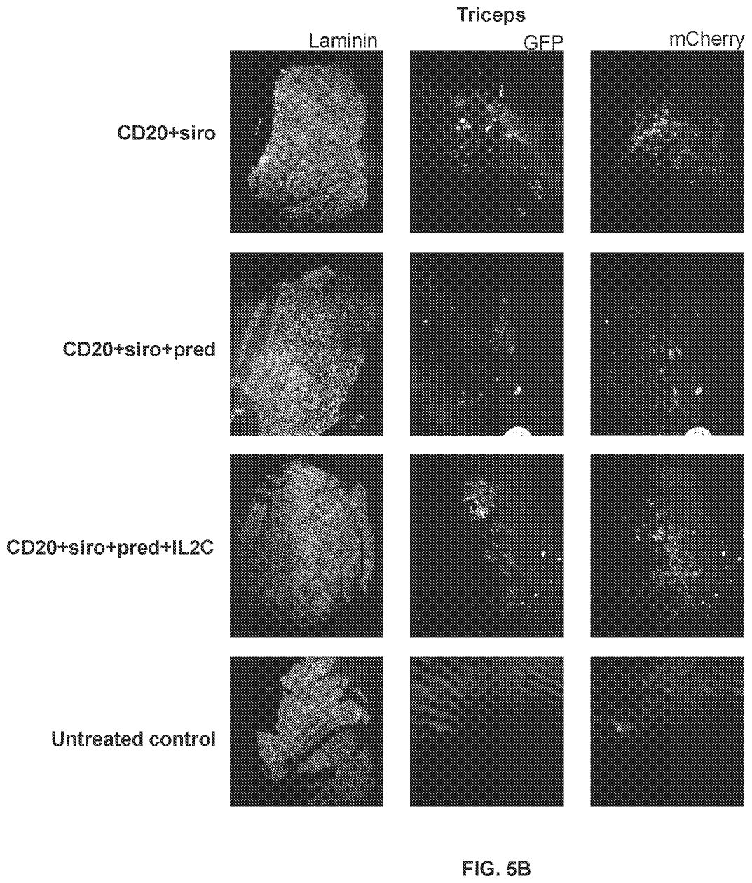

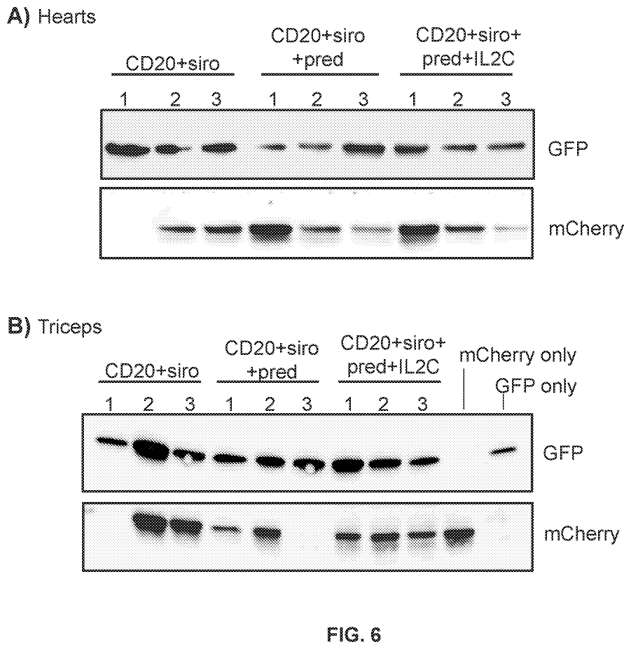

The following immunomodulation regimens were tested for their ability to allow for AAV redosing in vivo similar to above: 1) anti-CD20 antibody and sirolimus; 2) anti-CD20 antibody, sirolimus, and prednisone; 3) anti-CD20 antibody, sirolimus, prednisone, and IL2 complex. Control groups included prednisone only, no immune suppression, and untreated mice. The same timeline as FIG. 1 and dosing regimen in Table 2 was used in mdx mice 8-9 weeks old. On day 7, mice were retro-orbitally injected with AAV9-CMV-GFP and AAV9-Ck8-Cas9 at a concentration of 1.16×10¹⁴ vector genomes (vgs)/kg/vector (˜3×10¹² vgs/mouse/vector). About one month later, mice were given a retro-orbital injection of AAV9-target (containing mCherry and 45-55 gRNAs) and AAV9-Ck8-Cas9 at a concentration of 1.16×10¹⁴ vector genomes (vgs)/kg/vector (˜3×10¹² vgs/mouse/vector). About one month later, muscles were imaged, and Western blotted for GFP and mCherry as described above. All immunomodulatory regimens from groups 1-3, except one anti-CD20 antibody and sirolimus mouse (and one anti-CD20 antibody, sirolimus, prednisone mouse which did not have detectable mCherry in triceps but did in the heart), demonstrated redosing via expression of mCherry (FIGS. 5A, 5B, and 6).

### Example 4

### Redosing (Assessment of mCherry Expression) After Immunosuppressive Regimen Administration with AAV

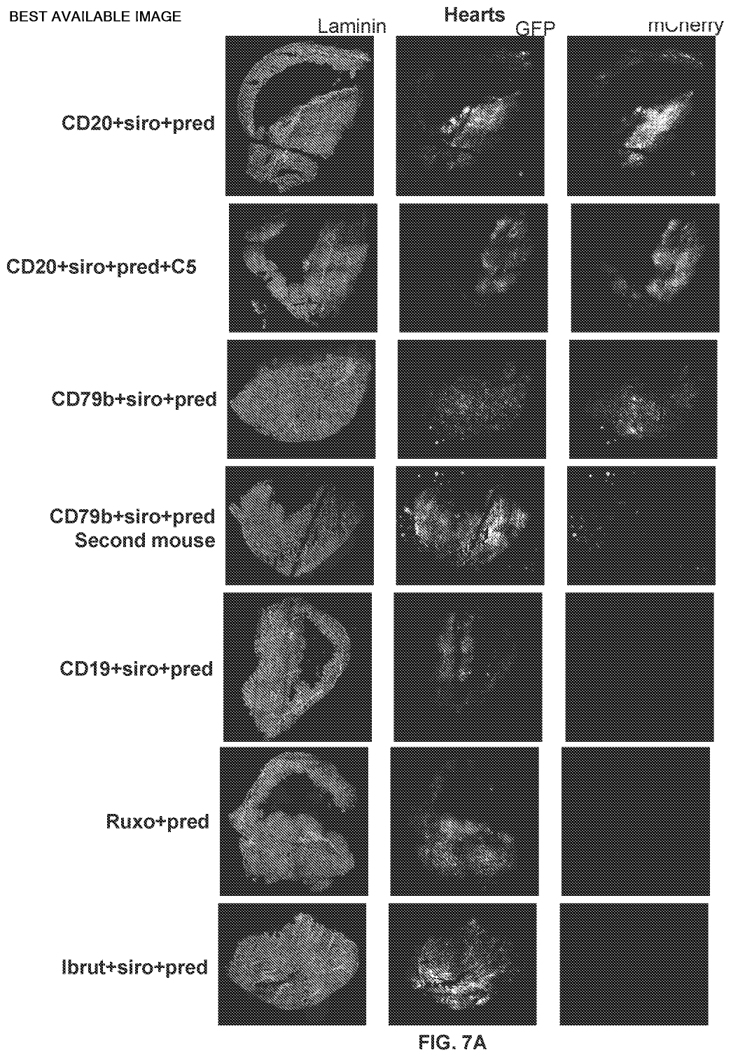

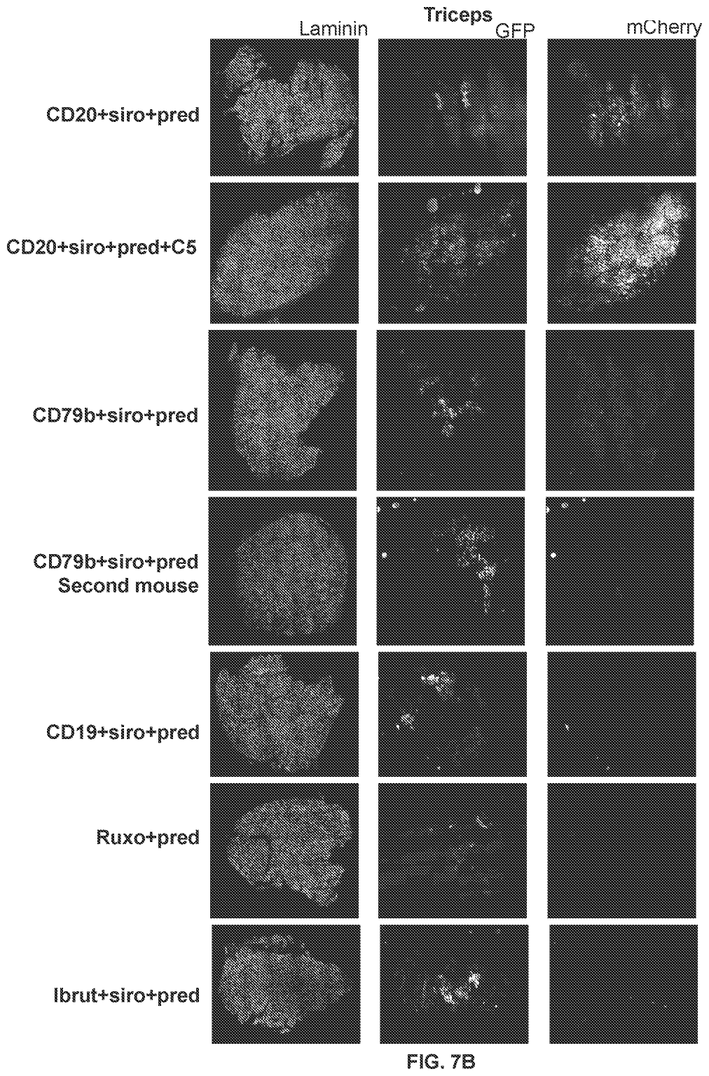

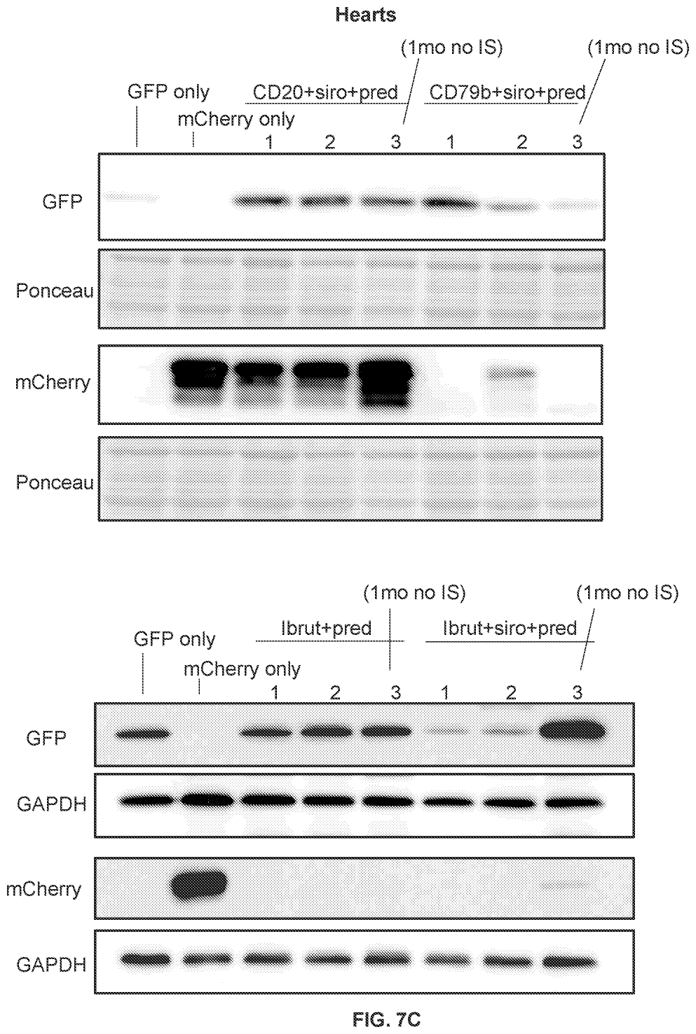

The following immunomodulation regimens were tested for their ability to allow for AAV redosing in vivo similar to above: 1) anti-CD20 antibody, sirolimus, and prednisone; 2) anti-CD20 antibody, sirolimus, prednisone, and anti-CS antibody; 3) anti-CD79b antibody, sirolimus, and prednisone; 4) anti-CD19 antibody, sirolimus, and prednisone; 5) ruxolitinib and prednisone; 6) ibrutinib, sirolimus, and prednisone. See Table 2 for dosing information on ruxolitinib, ibrutinib, anti-CD5 antibody, anti-CD79b antibody, and anti-CD19 antibody. Mdx mice at 6-8 weeks of age were treated according to the timeline in FIG. 1. On day 7, mice were retro-orbitally injected with AAV9-CMV-GFP and AAV9-Ck8-Cas9 at a concentration of 1.16×10¹⁴ vector genomes (vgs)/kg/vector (˜3×10¹² vgs/mouse/vector). About one month later, mice were given a retro-orbital injection of AAV9-target (containing mCherry and 45-55 gRNAs) and AAV9-Ck8-Cas9 at a concentration of 1.16×10¹⁴ vector genomes (vgs)/kg/vector (˜3×10¹² vgs/mouse/vector). About one month later, muscles were imaged for GFP and mCherry expression. Some mice were kept for an additional month after stopping immune suppression from observing if the redosing was long-lasting. The groups which showed redosing via expression of mCherry were: 1) anti-CD20 antibody, sirolimus, and prednisone; 2) anti-CD20 antibody, sirolimus, prednisone, and anti-CS antibody; and 3) one mouse that was given anti-CD79b antibody, sirolimus, and prednisone (FIGS. 7A, 7B, and 7C)

### Example 5

### Redosing AAV Carrying CRISPR/Cas9 in Combination with Immunosuppressive Regimen Administration

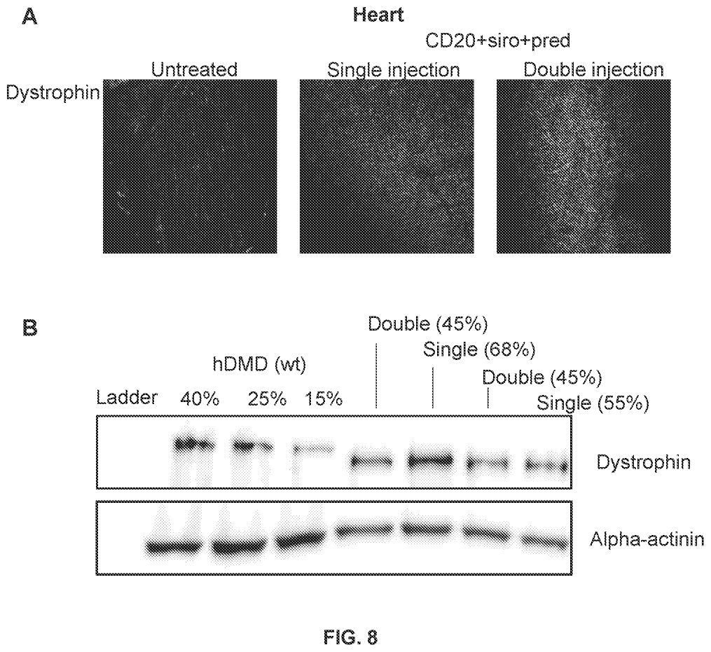

The immunosuppression regimen of anti-CD20 antibody, sirolimus, and prednisone was used to redose dual vector AAV9-CRISPR carrying Cas9 and gRNAs to delete exons 45-55 in humanized hDMD de145 mdx mice. Immune suppression was started at p7. Either one or two injections of dual AAV-CRISPR, comprising 1.5×10¹⁴ vg/kg/vector of AAV9-Ck8-SpCas9 and AAV9-target (containing 44C4, 55C3 gRNAs), were given at p14 and p21 if applicable. At around 8 weeks of age, the mice were sacrificed, and their muscles were assessed for dystrophin expression (FIG. 8).

Other redosing experiments using one, two, three, or more injections of AAV9-CRISPR with immune suppression are tested in hDMD de145 mdx mice. Immune suppression regimens start at p6-p7, and AAV-CRISPR is injected at p14, p17, and p21 as applicable. Additional redosing experiments using one, two, three, or more injections of AAV9-CRISPR with immune suppression are tested in juvenile hDMD de145 mdx mice starting at around 6 weeks of age. These immune suppression regimens include: 1) anti-CD20 antibody, sirolimus, and prednisone; 2) anti-CD20 antibody, sirolimus, prednisone, and IL2 complex; 3) anti-CD20 antibody, sirolimus, prednisone, and anti-05 antibody; and 4) CD79b antibody, sirolimus, and prednisone.

1. A method for treating muscular dystrophy in a patient in need thereof, comprising administering to the patient one or more doses of a particle comprising a recombinant adeno-associated viral (rAAV) nucleic acid vector, the vector comprising a polynucleotide that comprises a first nucleotide sequence that encodes a class 2 CRISPR/Cas endonuclease and one or more second nucleotide sequences, from which are transcribed a first and a second CRISPR/Cas9 guide RNA, wherein the first CRISPR/Cas9 guide RNA comprises a first guide sequence that hybridizes to a first target sequence within intron 44 of a mutant dystrophin gene in the patient, and the second CRISPR/Cas9 guide RNA comprises a second guide sequence that hybridizes to a second target sequence within intron 55 of the mutant dystrophin gene in the patient, and wherein administering the particle results in a deletion of a greater than 330 kb region of the mutant dystrophin gene comprising exons 45-55 in the patient.

2. A method for viral delivery of a nucleic acid in a patient in need thereof, comprising administering to the patient one or more doses of a particle comprising a recombinant adeno-associated viral (rAAV) nucleic acid vector, the vector comprising a polynucleotide that comprises a nucleic acid segment, wherein administering the particle to the patient results in deletion of a segment of a mutant gene in the patient.

3. The method of claim 2 or 3, wherein the treatment of muscular dystrophy comprises inhibiting progression of muscular dystrophy in a patient in need thereof.

4. The method of any one of claims 1-3, wherein the muscular dystrophy is Duchenne muscular dystrophy or Becker muscular dystrophy.

5. The method of any one of claims 1-4, wherein the class 2 CRISPR/Cas endonuclease is a type II CRISPR/Cas nuclease.

6. The method of claim 6, wherein the class 2 CRISPR/Cas endonuclease is a Cas9 protein, and the corresponding CRISPR/Cas guide RNA is a Cas9 guide RNA.

7. The method of claim 6, wherein the class 2 CRISPR/Cas endonuclease is a type V or type VI CRISPR/Cas endonuclease.

8. The method of claim 8, wherein the class 2, CRISPR/Cas endonuclease is chosen from Cpf1, C2c1, C2c3, and C2c2.

9. The method of any one of claims 1-8, wherein the first guide sequence comprises the 20-nucleotide sequence set forth in SEQ ID NO:3.

10. The method of any one of claims 1-9, wherein the second guide sequence comprises the 20-nucleotide sequence set forth in SEQ ID NO:7.

11. The method of any one of claims 1-10, wherein the first target sequence and the second target sequence are separated from each other by 500 kb or more.

12. The method of claim 11, wherein the first target sequence and the second target sequence are separated from each other by 700 kb or more.

13. The method of any one of claims 1-12, further comprising administering an immunosuppressive agent to the patient before administering the particle.

14. The method of claim 13, wherein the immunosuppressive agent is chosen from rapamycin, anti-CD20 antibody, rituximab, IL-2 complex, prednisone, anti-CD52 antibody, alemtuzumab, anti-CD19 antibody, anti-CD79 antibody, ibrutinib, mycophenolate mofetil, dimethyl fumarate, vamorolone, and complement inhibitor.

15. The method of claim 14, wherein the immunosuppressive agent is administered alone or in a combination.

16. The method of claim 15, wherein the immunosuppressive agent is administered in the combination comprising rapamycin, anti-CD20 antibody, and prednisone.

17. The method of claim 17, wherein the immunosuppressive agent is administered in a combination comprising rapamycin, anti-CD20 antibody, IL-2 complex, and prednisone.

18. The method of any one of claims 1-17, wherein the progression of Duchenne muscular dystrophy is inhibited or reversed for at least 50 days, at least 75 days, at least 100 days, at least 125 days, at least 150 days, at least 175 days at least 200 days, or more than 200 days after administering the particle.

19. The method of any one of claims 1-18, wherein the nucleic acid vector further comprises a promoter that is a muscle-specific promoter or a cytomegalovirus (CMV) promoter.

20. The method of claim 20, wherein the nucleic acid vector further comprises the muscle-specific promoter Ck8 promoter.

21. The method of any one of claims 1-20, wherein the particle is administered to the patient in a single injection.

22. The method of any one of claims 1-20, wherein the particle is administered to the patient in two injections.

23. The method of any one of claims 1-20, wherein the particle is administered to the patient in multiple injections.

24. The method of any one of claims 1-23, wherein the particle comprises the same serotype and is administered to the same patient.

25. The method of any one of claims 1-24, wherein a therapeutically-effective amount of the rAAV nucleic acid vector is an amount between 10⁶ and 10¹⁴ vector genomes (vgs)/kg.

26. A recombinant adeno-associated viral (rAAV) particle comprising a rAAV nucleic acid vector, the vector comprising a polynucleotide that comprises a first nucleotide sequence that encodes a class 2 CRISPR/Cas endonuclease and one or more second nucleotide sequences, from which are transcribed a first and a second CRISPR/Cas9 guide RNA, wherein the first CRISPR/Cas9 guide RNA comprises a first guide sequence that hybridizes to a first target sequence, and the second CRISPR/Cas9 guide RNA comprises a second guide sequence that hybridizes to a second target sequence within intron 55 of the mutant dystrophin gene.

27. The rAAV particle of claim 26, wherein the first target sequence is within intron 44 of a mutant dystrophin gene.

28. The rAAV particle of claim 26 or 27, wherein the first guide sequence comprises the 20-nucleotide sequence set forth in SEQ ID NO:3.

29. The rAAV particle of any one of claims 26-28, wherein the second target sequence is within intron 55 of a mutant dystrophin gene.

30. The rAAV particle of any one of claims 26-29, wherein the second guide sequence comprises the 20-nucleotide sequence set forth in SEQ ID NO:7.

31. The rAAV particle of any one of claims 26-30, wherein the first target sequence and the second target sequence are separated from each other by 500 kb or more.

32. The rAAV particle of claim 31, wherein the first target sequence and the second target sequence are separated from each other by 700 kb or more.

33. The rAAV particle of any one of claims 26-32, wherein the class 2 CRISPR/Cas endonuclease is a type II CRISPR/Cas nuclease.

34. The rAAV particle of any one of claims 26-32, wherein the class 2 CRISPR/Cas endonuclease is a Cas9 protein, and the corresponding CRISPR/Cas guide RNA is a Cas9 guide RNA.

35. The rAAV particle of any one of claims 26-32, wherein the class 2 CRISPR/Cas endonuclease is a type V or type VI CRISPR/Cas endonuclease.

36. The rAAV particle of any one of claims 26-32, wherein the class 2, CRISPR/Cas endonuclease is chosen from Cpf1, C2c1, C2c3, and C2c2.

37. The rAAV particle of any one of claims 26-36, wherein administering the particle to a patient in need thereof results in a deletion of a greater than 330 kb region of the mutant dystrophin gene comprising exons 45-55 in the patient.

38. A pharmaceutical composition comprising a plurality of rAAV particles of any one of claims 26-37 and a carrier.

39. A method for treating muscular dystrophy in a patient in need thereof, comprising administering to the patient a plurality of rAAV particles of any one of claims 26-37.

40. A method for treating muscular dystrophy in a patient in need thereof, comprising administering to the patient a pharmaceutical composition of claim 38.


SEQUENCE LISTING (8 sequences)

SEQ ID NO: 1 - DNA, 20 bp, Streptococcus pyogenes
gttgaaattaaactacacac

SEQ ID NO: 2 - DNA, 17 bp, Streptococcus pyogenes
gaaattaaactacacac

SEQ ID NO: 3 - RNA, 20 nt, Streptococcus pyogenes
guugaaauuaaacuacacac

SEQ ID NO: 4 - RNA, 17 nt, Streptococcus pyogenes
gaaauuaaacuacacac

SEQ ID NO: 5 - DNA, 20 bp, Streptococcus pyogenes
tgtatgatgctataatacca

SEQ ID NO: 6 - DNA, 17 bp, Streptococcus pyogenes
atgatgctataatacca

SEQ ID NO: 7 - RNA, 20 nt, Streptococcus pyogenes
uguaugaugcuauaauacca

SEQ ID NO: 8 - RNA, 17 nt, Streptococcus pyogenes
augaugcuauaauacca